(19) United States
(12) Patent Application Publication
(10) Pub. No.: US 2004/0028737 A1
(43) Pub. Date: Feb. 12, 2004

(54) Enteric coated stable oral pharmaceutical composition of acid unstable drug and process for preparing the same

(75) Inventors: Jayant Venkatesh Deshpande, Maharashtra (IN); Vandana Sandeep Gupte, Maharashtra (IN); Vaishali Madhukar Kadam, Maharashtra (IN); Chandrakant Thakarsi Gosar, Maharashtra (IN); Satish Ramachandra Deshmukh, Maharashtra (IN); Rajan Vitthal Gupte, Maharashtra (IN); Vijay Ramachandra Tamhankar, Maharashtra (IN)

Correspondence Address: GREENBLUM & BERNSTEIN, P.L.C., 1950 ROLAND CLARKE PLACE, RESTON, VA, 20191, US

(73) Assignee: Kopran Research Laboratories Limited, Maharashtra (IN)

(21) Appl. No.: 10/216,315

(22) Filed: Aug. 12, 2002

(51) Int. Cl. A61K 31/4439; A61K 9/16; A61K 9/50

(52) U.S. Cl. 424/474; 514/338

(57) ABSTRACT

Enteric coated stable oral pharmaceutical composition of acid unstable drug. The enteric coating is a bilayer with a pH gradient across its thickness comprising an inner layer of neutral or near neutral pH 7-7.5 and an outer layer of acidic pH 2-6. Also process for preparng the enteric coated stable oral pharmaceutical composition of acid unstable drug. The enteric coating is first carried out at neutral or near neutral pH of 7-7.5 to form an inner layer of neutral or near neutral pH and then at acidic pH of 2-6 to form an outer layer of acidic pH.

### FIELD OF INVENTION

Enteric coated stable oral pharmaceutical composition of acid unstable drug and process for preparing the same.

### PRIOR ART

Oral pharmaceutical compositions comprising acid unstable drugs (medicaments) are enteric coated to render them acid compatible and prevent decomposition or destruction thereof due to the harmful effects of acid secreted by stomach and to improve the storage stability thereof. Such drugs cannot be, however, directly enteric coated, as most of the enteric substances are also acidic in nature. Therefore, a subcoating or barrier costing is provided or alternatively compounds like alkaline salts, fine amino acids, polyvinyl pyrrolidone or mannitol is used along with the drugs. Several enteric coated acid labile drugs are reported in the literature.

U.S. Pat. No. 4,786,505 of Lovgren et al describes an omeprazole based oral pharmaceutical preparation in combination with alkaline reacting compound provided with a subcoating of excipients and polymeric film forming compounds and an outer enteric coating.

U.S. Pat. No. 5,026,560 of Makino et al teaches spherical granules having a core coated with spraying powder containing a drug such as benzimidazole and a binder and further coated with enteric material.

U.S. Pat. No. 5,626,875 of Ballester et at relates to benzimidazole based pharmaceutical preparation comprising an inert non-alkaline coating and an outer enteric coating.

U.S. Pat. No. 6,224,910 of Ullah et al teaches a pharmaceutical composition of a medicament sensitive to low pH such as didanosine comprising an enteric coating and an additional anti-adherent coating.

U.S. Pat. No. 6,346,269 of Hsiao et al describes an oral pharmaceutical formulation comprising an acid sensitive drug such as omeprazole spread onto a core along with a stabilizer and subcoated with an adhesive and finally enteric coated.

Due to the dual coatings employed in the above U.S. Patents, the time, labour and cost of preparation of the pharmaceutical compositions increase thereby correspondingly increasing the cost of the compositions. Besides, because of the non-enteric coating, absorption of the drugs in the intestines may be reduced. Over a period of time the enteric coating may penetrate the drugs and decompose or destabilise them thereby reducing the shelf life of the drugs.

U.S. Pat. Nos. 5,045,321 and 5,093,132 of Makino et al relate to stabilised pharmaceutical composition of benzimidazoles with basic inorganic salt stabilising agent such as salts of magnesium and/or calcium provided with an enteric coating.

U.S. Pat. No. 5,385,739 of Debregeas et al describes stable formulation of omeprazole micro granules characterized by an active layer of omeprazole in mannitol in substantially equal amounts and provided with enteric coating.

U.S. Pat. No. 6,013,281 of Lundberg et al relates to an oral pharmaceutical formulation of a proton pump inhibitor (PPI) such as omeprazole and at least one alkaline reacting compound provided with an enteric coating.

U.S. Pat. No. 6,068,856 of Sachs et al teaches an oral pharmaceutical composition of pantoprazole in pellet or tablet form provided with a slow release coat and an enteric coating.

U.S. Pat. No. 6,096,340 of Chen et al describes a pharmaceutical composition of omeprazole in combination with surface active agent filler, pharmaceutically acceptable alkaline agent and binder provided with an enteric coating.

U.S. Pat. No. 6,248,758 of Klokkers et al relates to a pharmaceutical formulation of benzimidazole with excipients, cyclodextrin and at least one amino acid provided with an enteric coating.

U.S. Pat. No. 6,331,316 of Ullah et al describes a pharmaceutical tablet comprising a core comprising 2,3′-dideoxyinosine (ddl), binder or filler, disintegrant, and lubricant. The core is coated with an enteric coating comprising a methacrylic acid copolymer and a plasticizer having a pH of 5.0±0.1.

European Patent No 0960620 (WO 99/61022) of Ranbaxy Laboratories relates to a pharmaceutical composition of substituted pyridyl sulfinyl benzimidazole and pharmaceutically acceptable carriers comprising at least one polymer having vinyl pyrrolidone monomeric units and provided with enteric coating.

In the case of the above pharmaceutical compositions comprising only a coating of enteric materials the enteric coating penetrates the core and degrades or decomposes the drugs over a period of time under ambient storage conditions. Therefore, such compositions do not have long shelf life and they are to be administered within a short time after their manufacture. Processes for the preparation of such compositions, however, require comparatively less time, labour and cost and are economical thereby reducing the cost of the compositions correspondingly.

### OBJECTS OF INVENTION

An object of the invention is to provide enteric coated stable oral pharmaceutical composition of acid unstable drug which is economical.

Another object of the invention is to provide enteric coated stable oral pharmaceutical composition of acid unstable drug which has long storage life.

Another object of the invention is to provide enteric coated stable oral pharmaceutical composition of acid unstable drug which has excellent dissolution rate in the intestinal fluid.

Another object of the invention is to provide enteric coated stable oral pharmaceutical composition of acid unstable drug which does not have a barrier coating and does not make use of compounds like alkaline salts, alkaline amino acids, polyvinyl pyrrolidone or mannitol.

Another object of the invention is to provide a process for preparing enteric coated stable oral pharmaceutical composition of acid unstable drug, which is simple and easy to carryout and requires comparatively less time, labour and cost and is economical.

Another object of the invention is to provide a process for preparing enteric coated stable oral pharmaceutical composition of acid unstable drug, which does not employ a barrier coating and compounds like alkaline salts, alkaline amino acids, polyvinyl pyrrolidone or mannitol.

### DETAILED DESCRIPTION OF INVENTION

According to the invention there is provided enteric coated stable oral pharmaceutical composition of acid unstable drug in combination with pharmaceutically acceptable excipients and coated with an enteric material wherein the enteric coating is a bilayer with a pH gradient across its thickness comprising an inner layer of neutral or near neutral pH 7-7.5 and an outer layer of acidic pH 2-6.

Preferably the neutral or near neutral pH inner layer comprises up to ¼ th of the enteric coating thickness and the acidic pH outer layer comprises up to ¾th of the enteric coating thickness.

According to the invention there is also provided process for preparing enteric coated stable oral pharmaceutical composition of acid unstable drug comprising formulating the acid unstable drug with pharmaceutically acceptable excipients followed by enteric coating the formulation with an enteric material to provide a bilayer with a pH gradient by first carrying out the enteric coating at neutral or near neutral pH of 7-7.5 to form an inner layer of neutral or near neutral pH and then carrying out the enteric coating at acidic pH of 2-6 to form an outer layer of acidic pH.

Generally enteric coating materials like methacrylate copolymers or hydroxypropyl methyl cellulose phthalate (HPMCP) are coated by spraying through a nozzle either as an aqueous dispersion at pH 2-5.5 or as a solution in organic solvent(s). It is known that if the pH of the aqueous dispersion of the polymers is raised above 6, the polymer gets dissolved and the solution becomes extremely viscous and cannot be sprayed through the nozzle. Due to absence of H⁺ ions, pH of the solution of the polymers in organic solvents cannot be adjusted. Surprisingly it has been found that using an aqueous organic diversion of the enteric material in certain proportion as described in the specification it is possible to raise the pH of the dispersion to neutral or near neutral pH and carryout the enteric coating to provide an enteric coating of neutral or near neutral pH without increasing the viscosity of the solution and without loosing the film forming property of the polymer.

According to the invention the acid sensitive drug may be benzimidazole derivatives such as omeprazole, pantoprazole, lansoprazole or rabeprazole or salts or optical isomers/enantiomers thereof.

According to the invention, the acid sensitive drug also may be penicillin, methicillin, erythromycin and its derivatives, carbenicillin, antifungal agents such as ketoconazole or itraconazole, enzymes such as pancreatin, levodopa didanosine, pravastatin or digoxin, proteins or peptides such as insulin.

The excipients may be alkaline or non-alkaline and may be binders such as starch, gelatin, sugars such as sucrose, glucose, dextrose, molasses or lactose, natural or synthetic gums like acacia, sodium alginate, carboxy methyl cellulose, methyl cellulose, poly vinyl pyrrolidone) polyethylene glycol, ethyl cellulose, waxes, corn starch or hydroxypropyl methyl cellulose or fillers like sugars such as lactose, dextrose, sucrose, maltose, microcrystalline cellulose or calcium sulphate or lubricants such as sodium stearyl fumarate, magnesium stearate, talc, calcium stearate, stearic acid, hydrogenated vegetable oils or polyethylene glycol or glidants such as colloidal silicon dioxide or talc or diluents such as dicalcium phosphate, calcium sulphate, lactose, cellulose, kaolin, mannitol, sodium chloride, dry starch, powdered sugar, light silicic anhydride, sorbitol or inositol or whiteners such as titanium dioxide or Opadry. The excipients may include surfactants such as sodium lauryl sulphate, tween 80 or sodium dodecyl benzene sulphonate and/or plasticisers such as triacetin, citric acid esters, phthalic acid esters, dibutyl sebacate, dibutyl succinate, cetyl alcohol, polyethylene glycols, polysorbates, stearyl alcohol citroflex or dimethyl, polysiloxan.

The pharmaceutical compositions of the invention may be particles, pellets, granules or tablets or capsules containing the enteric coated particles, pellets or granules.

The first enteric coating at neutral or near neutral pH may be carried out to form an inner layer up to ¼th of the enteric coating thickness and the subsequent enteric coating at acidic pH is carried out to form an outer layer up to ¾th of the enteric coating thickness.

The enteric coating at neutral or near neutral pH may be carried out using an aqueous organic dispersion of the enteric material comprising water and water miscible organic solvent in the ratio 10:90-5:95 v/v and 4-10% by weight of the enteric material.

The water miscible organic solvent may be alcohol such as methanol, ethanol or isopropyl alcohol, acetone or acetonitrile preferably isopropyl alcohol.

The enteric coating at neutral or near neutral pH may be caned out preferably using aqueous organic dispersion of the enteric material comprising water and isopropyl alcohol in the ratio 8:92 v/v and 6% by weight of the enteric material such as methacrylate copolymer Type C USP/NF.

The enteric coating at acidic pH may be carried out using an aqueous dispersion of the enteric material comprising 6 to 10% by weight of the enteric material.

The enteric coating at acidic pH may be carried out preferably using an aqueous dispersion of the enteric material comprising 8% by weight of methacrylate copolymer Type C USP/NF.

The acidic pH may be adjusted with an alkali such as sodium hydroxide, potassium hydroxide, aluminum hydroxide, calcium hydroxide, magnesium hydroxide, light magnesium carbonate or disodium hydrogen phosphate preferably sodium hydroxide.

The enteric material may be polymers such as solutions or dispersions of methacrylic acid copolymers such as methacrylate polymer type C USP/NF, cellulose acetate phthalate, hydroxypropyl methyl cellulose phthalate, hydroxypropyl methylcellulose acetate succinate, polyvinyl acetate phthalate, cellulose acetate trimellitate, carboxymethyl ethyl cellulose or shellac, preferably methacrylate copolymer type C USP/NF.

According to the invention the pharmaceutical composition comprises only one enteric coating comprising a neutral or near neutral pH inner layer and acidic pH outer layer. The neutral or near neutral pH inner layer is compatible with the acid labile drug and protective of the drug against the acidic pH outer layer. Therefore, the pharmaceutical composition of the invention has long shelf life and stability under ambient storage conditions. The acidic pH outer layer serves as an enteric film and is compatible with and protects the acid labile drug against the stomach acids. Both the neutral or near neutral inner layer and acidic outer layer of the enteric coating are easily soluble in the alkaline intestinal environment thereby rapidly releasing the drug into the intestines. According to the invention, the barrier coating and compounds like alkaline salts, alkaline amino acids, polyvinyl pyrrolidone and mannitol have been eliminated. Therefore, the process of the invention is simple and comparatively less time consuming, less labour oriented and cumbersome aid less costly. The composition of the invention is also comparatively less costly.

The following experimental examples are illustrative of the invention but not limitative of the scope thereof.

### EXAMPLE 1

Omeprazole tablets of the following composition were prepared

| | | |
|---|---|---|
| Omeprazole | 10.30 | mg |
| Lactose anhydrous | 55.00 | mg |
| Magnesium stearate | 1.00 | mg |
| Talc | 1.00 | mg |
| Colloidal silicon dioxide | 0.50 | mg |
| Microcrystalline cellulose | 17.00 | mg |
| Maize starch | 10.00 | mg |
| Povidone (PVP-K-30) | 3.00 | mg |

Omeprazole was mixed with lactose anhydrous, colloidal silicon dioxide, microcrystalline cellulose and maize starch and granulated with povidone dissolved in water. Wet granules were passed through sieve 12 and dried in a tray vacuum dryer at 30° C. for 10 hours. The dried granules were passed through sieve 12 and mixed with talc and magnesium stearate and compressed into tablets.

The tablets were enteric coated with the following aqueous organic dispersion of enteric coating material at neutral pH 7

| | | |
|---|---|---|
| Methacrylate copolymer Type C USP/NF | 0.4 | kg |
| Isopropyl alcohol | 4.0 | lit |
| Purified Water | 0.375 | lit |
| Polysorbate 80 | 0.02 | kg |
| PEG (Polyethylene glycol) 600 | 0.04 | kg |
| Titanium dioxide | 0.05 | kg |
| Talc | 0.165 | kg |

The methacrylate copolymer Type C USP/NF was suspended in a mixture of isopropyl alcohol and water (90/o v/v). The pH of the solution was adjusted to neutrality, 7, using 2M ammonia solution. It was then mixed with polysorbate 80, PEG 600, titanium dioxide and talc. Coating was carried out in a coating pan until the tablets gained a weight of 4%. Thereafter the coating was continued farther in an aqueous dispersion of methacrylate copolymer Type C USP (USP/NF 23, Page 2478) containing 3% w/v castor oil and 1% of purified talc at pH 3.0 till a weight gain of 12% by the tablets.

### EXAMPLE 2

Lansoprazole pellets of the following composition were prepared:

| | | |
|---|---|---|
| Non pereil seeds (sugar) | 0.91 | kg |
| Lansoprazole | 0.154 | kg |
| Microcrystalline cellulose | 0.1 | kg |
| Starch | 0.08 | kg |
| Purified water | 1.1 | kg |

Lansoprazole and microcrystalline cellulose were suspended in the solution of starch in the purified water and sprayed on the non pereil seeds of sugar in a coating pan.

The pellets (1 kg) were enteric coated with the following aqueous organic dispersion of enteric coating material at neutral pH 7:

| | | |
|---|---|---|
| Methacrylate copolymer Type C USP/NF | 0.4 | kg |
| Isopropyl alcohol | 4.0 | lit |
| Purified water | 0.375 | lit |
| Polysorbate 80 | 0.02 | kg |
| PEG (polyethylene glycol) 600 | 0.04 | kg |
| Titanium dioxide | 0.05 | kg |
| Talc | 0.165 | kg |

The methacrylate copolymer Type C USP/NF was suspended in a mixture of isopropyl alcohol and water (90% v/v). The pH of the solution was adjusted to neutrality 7 using 2M ammonia solution. It was then mixed with polysorbate 80, PEG 600, titanium dioxide and talc. The pellets were coated in a coating pan with above dispersion till weight gain of 6%. Thereafter, coating was continued further in an aqueous dispersion of methacrylate copolymer Type C (USP/NF 23, Page 2478), containing 3% w/v castor oil and 1% talc at pH 3.0 till a weight gain of 20%.

### EXAMPLE 3

Pantoprazole tablets of the following composition were prepared

| | | |
|---|---|---|
| Pantoprazole sodium | 43.5 | mg |
| Microcrystalline cellulose | 20 | mg |
| Starch soluble | 35 | mg |
| Polyvinyl pyrrolidone | 4 | mg |
| Magnesium stearate | 1 | mg |
| Talc | 1 | mg |

Pantoprazole sodium was mixed with microcrystalline cellulose and starch and granulated with polyvinyl pyrrolidone solution in purified wafer. Wet granules were passed through sieve 12 and dried in a tray vaccum dryer at 30° C. for 10 hours. The dried granules were passed through sieve 12 and mixed with talc and magnesium stearate and compressed into tablets.

The tablets are enteric coated with the following aqueous organic dispersion of enteric coating material at near neutral pH 7.5.

| | | |
|---|---|---|
| Methacrylate copolymer Type C USP/NF | 0.4 | kg |
| Isopropyl alcohol | 4.0 | lit |
| Purified water | 0.375 | lit |
| Polysorbate 80 | 0.02 | kg |
| PEG (polyethylene glycol) 600 | 0.04 | kg |
| Titanium dioxide | 0.05 | kg |
| Talc | 0.145 | kg |

The methacrylate copolymer Type C USP/NF was suspended in a mixture of isopropyl alcohol-water (90% v/v). The pH of the solution was adjusted to near neutrality 7.5 using 2M ammonia solution. It was then mixed with polysorbate 80, PEG 600, titanium dioxide and talc. The tablets were coated in a coating pan with the above dispersion till a weight gain of 4%. Thereafter, the coating was continued further in an aqueous dispersion of methacrylate copolymer Type C (USP/NF 23, Page 2478), containing 100% w/v castor oil and 4% talc at pH 5.0 using 2N sodium hydroxide till a weight gain of 12%.

### EXAMPLE 4

Lansoprazole pellets of the following composition were prepared:

| | | |
|---|---|---|
| Non pereil seeds (Sugar) | 0.91 | kg |
| Lansoprazole | 0.154 | kg |
| Disodium hydrogen phosphate | 0.012 | kg |
| Light Magnesium carbonate | 0.085 | kg |
| Starch | 0.08 | kg |
| Microcrystalline cellulose | 0.1 | kg |
| Purified water | 1.1 | kg |

Lansoprazole, microcrystalline cellulose, light magnesium carbonate and disodium hydrogen phosphate were suspended in the solution of starch and purified water and sprayed on the non pereil seeds of sugar in a costing pan.

1 kg of pellets were enteric coated in a coating pan with the following aqueous organic dispersion of enteric coating material at neutral pH 7:

| | | |
|---|---|---|
| Methacrylate copolymer Type C USP/NF | 0.4 | kg |
| Isopropyl alcohol | 4.0 | lit |
| Purified water | 0.375 | lit |
| Polysorbate 80 | 0.02 | kg |
| PEG (polyethylene glycol) 600 | 0.04 | kg |
| Titanium dioxide | 0.05 | kg |
| Talc | 0.165 | kg |

The methacrylate copolymer Type C USP/NF was suspended in a mixture of isopropyl alcohol and water (900% v/v). The pH of the solution was adjusted to neutrality 7 using 2M ammonia solution. It was then nixed with polysorbate 80, PEG 600, tianium dioxide and talc. The pellets were coated in a coating pan, till a weight gain of 6%. Thereafter coating was continued further in an aqueous dispersion of methacrylate copolymer Type C (USP/NF 23, Page 2478) containing 10% PEG 600 at pH 5.5 till a weight gain of 20%.

### EXAMPLE 5

Uncoated omeprazole tablets were prepared as described in Example 1 and given a protective barrier coat with the following solution:

| | | |
|---|---|---|
| Polyvinylpyrrolidone K-30 | 0.1 | kg |
| Talc | 0.1 | kg |
| PEG (Polyethylene glycol) | 0.89 | kg |
| Purified water | 1.01 | kg |

The omeprazole tablets were coated with the solution in a coating pan till a weight gain of 4%. The tablets were dried in a vacuum tray dryer at 30° C. for 12 hours.

The tablets were given enteric coat with an aqueous dispersion of methacrylate copolymer Type C (USP/NF 23, page 2478) at pH 3.0 containing 3% w/v castor oil amid 1% by weight purified talc, till weight gain of 12% by weight.

### EXAMPLE 6

Lansoprazole loaded sugar seeds were prepared as per Example 2. 1 kg of lansoprazole loaded sugar beads were given a barrier coat with the following solution:

| | | |
|---|---|---|
| Hydroxy propyl methyl cellulose | 0.1 | kg |
| PEG (Polyethylene glycol) 6000 | 0.095 | kg |
| Talc | 0.11 | kg |
| Purified water | 1.21 | kg |

The tablets were coated with the solution in a coating pan till a weight gain of 6%. The tablets were dried in a vacuum drier at 30° C. for 10 hrs. The dried pellets were given an enteric coat in a coating pan with an aqueous dispersion of methacrylate copolymer Type C (USP/NF 23, Page 2478) containing 3% w/v castor oil and 2% by weight purified talc at pH 3.0 till a weight gain of 20%.

### EXAMPLE 7

Uncoated pantoprazole sodium tablets were prepared as per Example 3. 1.0 kg of pantoprazole tablets were given a barrier coating in a coating pan with the following coating solution:

| | | |
|---|---|---|
| Hydroxypropyl methyl cellulose | 0.116 | kg |
| PEG (Polyethylene glycol) 6000 | 0.095 | kg |
| Talc | 0.085 | kg |
| Purified water | 1.15 | kg |

The coating was performed till a weight gain of 4%. The tablets were dried in a vacuum drier at 30° C. for 12 hrs. The dried pellets were enteric coated with an aqueous dispersion of methacrylate copolymer Type C (USP/NF 23, Page 2478) containing 3% w/v castor oil and 2% purified talc at pH 5.0 using 2N sodium hydroxide till a weight gain of 12% by weight.

### EXAMPLE 8

Rabeprazole tablets of the following composition were prepared.

| | | |
|---|---|---|
| Rabeprazole | 10.30 | mg |
| Lactose anhydrous | 55.00 | mg |
| Magnesium stearate | 1.00 | mg |
| Talc | 1.00 | mg |
| Colloidal silicon dioxide | 0.50 | mg |
| Microcrystalline cellulose | 17.00 | mg |
| Maize starch | 10.00 | mg |
| Povidone (PVP-K-30) | 3.00 | mg |

Rabeprazole was mixed with lactose anhydrous, colloidal silicon dioxde, microcrystalline cellulose and maize starch and granulated with povidone dissolved in water. Wet granules were passed through sieve 12 and dried in a tray vacuum dryer at 30° C. for 10 hours. The dried granules were passed through sieve 12 and mixed with talc and magnesium stearate and compressed into tablets.

The tablets were enteric coated with the following aqueous organic dispersion of enteric coating material at neutral pH 7;

| | | |
|---|---|---|
| Methacrylate copolymer Type C USP/NF | 0.4 | kg |
| Isopropyl alcohol | 4.0 | lit |
| Purified Water | 0.375 | lit |
| Polysorbate 80 | 0.02 | kg |
| PEG (Polyethylene glycol) 600 | 0.04 | kg |
| Titanium dioxide | 0.05 | kg |
| Talc | 0.165 | kg |

The methacrylate copolymer Type C USP/NF was suspended in a mixture of isopropyl alcohol-water (90% v/v). The pH of the solution was adjusted to neutrality 7 using 2M ammonia solution. It was then mixed with polysorbate 80, PEG 600, titanium dioxide and talc. Coating was carried out in a coating pall until the tablets gained a weight of 4%. Thereafter the coating was continued further in an aqueous dispersion of methacrylate copolymer Type C USP/NF (USP/NF 23, Page 4278) containing 3% w/v castor oil and 1% of purified talc at pH 3.0 till a weight gam of 12% by the tablets.

### EXAMPLE 9

Uncoated rabeprazole tablets were prepared as described in Example 8 and given a barrier coating with the following solution:

| | | |
|---|---|---|
| Polyvinylpyrrolidone K-30 | 0.1 | kg |
| Talc | 0.1 | kg |
| PEG (Polyethylene glycol) | 0.89 | kg |
| Purified water | 1.01 | kg |

The rabeprazole tablets were coated with the solution in a coating pan till a weight gain of 4% and dried in a vacuum tray dryer at 30° C. for 12 hours.

The tablets were given enteric coating with an aqueous dispersion of methacrylate copolymer Type C (USP/NF 23, page 2478) at pH 3.0 containing 3%1% w/v castor oil and 1% by weight purified talc, till weight gain of 12% by weight.

Release Studies

Acid resistance study of the pharmaceutical products of Examples 1 to 9 was conducted by using the USP XXII dissolution test (Type I, basket) at 100 RPM 37° C. in an aqueous solution of HCl at pH 1.0. After 2 hrs, the products were filtered and tested for the assay of active pharmaceutical ingredient.

Dissolution at pH 6.8 was carried out in a separate study after treating the products for 2 hrs with 500 ml of 0.1N HCl at 37° C. The test samples were tested according to USP XXII dissolution test at 37° C. and 100 rpm in phosphate buffer at pH 6.8 to determine the drug dissolved in 30 min.

The results were as shown in the following Tables 1 to 4.

TABLE 1

| | Acid resistant (Assay | Dissolution at pH |
|---|---|---|
| | after acid treatment) | 6.8 (% released) in |
| Product | (%) | 30 min |
| Omeprazole tablets of | 96.56 | 94.54 |
| Example 1 | | |
| Omeprazole tablets of | 92.23 | 91.46 |
| Example 5 | | |

TABLE 2

| | Acid resistant (Assay | Dissolution at pH |
|---|---|---|
| | after acid treatment) | 6.8 (% released) in |
| Product | (%) | 30 min |
| Lansoprazole pellets of | 97.17 | 95.26 |
| Example 2 | | |
| Lansoprazole pellets of | 97.31 | 96.28 |
| Example 4 | | |
| Lansoprazole pellets of | 92.69 | 91.33 |
| Example 6 | | |

TABLE 3

| | Acid resistant (Assay | Dissolution at pH |
|---|---|---|
| | after acid treatment) | 6.8 (% released) in |
| Product | (%) | 30 min |
| Pantoprazole tablets of | 95.37 | 94.33 |
| Example 3 | | |
| Pantoprazole tablets of | 90.13 | 91.61 |
| Example 7 | | |

TABLE 4

| | Acid resistant (Assay | Dissolution at pH |
|---|---|---|
| | after acid treatment) | 6.8 (% released) in |
| Product | (%) | 30 min |
| Rabeprazole tablets of | 96.55 | 96.24 |
| Example 8 | | |
| Rabeprazole tablets of | 91.60 | 90.52 |
| Example 9 | | |

### CONCLUSION

Products of examples 1-4 and 8 have shown higher resistance to acid penetration compared to products of examples 5-7 and 9. Rate of dissolution of products of examples 1-4 and 8 is higher as compared to products of examples 5-7 and 9. Less than 10% of products of examples 1-4 and 8 was released in simulated gastric fluid whereas more than 90% were released in simulated intestinal fluid.

Stability Studies

Products of examples 1-4 and 8 and products of examples 5-7 and 9 were subjected to stability study at 40° C. and 75% RH (relative humidity) for 3 months. Stability was assessed in terms of release after 2 hrs treatment in acidic medium, followed by 30 mins treatment at pH 6.8 at the end of each month. The results were as shown in the following Table 5.

TABLET 5

| | % Release after storage at 40° C., 75% RH | | | |
|---|---|---|---|---|
| Product | Initial | 1 month | 2 month | 3 month |
| Omeprazole tablets | 94.57 | 94.41 | 93.82 | 93.53 |
| of Example 1 | | | | |
| Lansoprazole pellets of | 95.22 | 95.06 | 94.64 | 94.40 |
| Example 2 | | | | |
| Pantoprazole tablets of | 94.34 | 94.19 | 93.83 | 93.53 |
| Example 3 | | | | |
| Lansoprazole pellets of | 96.31 | 96.11 | 95.67 | 94.62 |
| Example 4 | | | | |
| Omeprazole tablets of | 91.46 | 90.68 | 89.53 | 89.14 |
| Example 5 | | | | |
| Lansoprazole pellets of | 91.33 | 91.14 | 90.81 | 90.52 |
| Example 6 | | | | |
| Pantoprazole tablets of | 91.60 | 89.65 | 89.16 | 89.01 |
| Example 7 | | | | |
| Rabeprazole Tablets | 96.24 | 96.18 | 96.10 | 95.88 |
| of Example 8 | | | | |
| Rabeprazole Tablets | 91.52 | 89.60 | 89.15 | 88.66 |
| of Example 9 | | | | |

### CONCLUSION

Products as per examples 1-4 and 8 had shown better storage stability when stored at 40° C. and 75% RH compared to products of examples 5-7 and 9. Products of examples 1-4 and 8 maintained their colour and appearance during the 3 months.

1) Enteric coated stable oral pharmaceutical composition of acid unstable drug in combination with pharmaceutically acceptable excipients and coated with an enteric material wherein the enteric coating is a bilayer with a pH gradient across its thickness comprising an inner layer of neutral or near neutral pH 7-7.5 and an outer layer of acidic pH 2-6.

2) Enteric coated stable oral pharmaceutical composition as claimed in claim 1, wherein the neutral or near neutral pH inner layer comprises up to ¼ th of the enteric coating thickness and the acidic pH outer layer comprises up to ¾th of the enteric coating thickness.

3) Enteric coated stable oral pharmaceutical composition as is claimed in claim 1 wherein the acid unstable drug is benzimidazole derivatives such as omeprazole, pantoprazole, lansoprazole or rabeprazole or salts or optical isomers/enantiomers thereof.

4) Enteric coated stable oral pharmaceutical composition as claimed in claim 1, wherein the acid unstable drug is penicillin, methicillin, erythromycin and its derivatives, carbenicillin antifungal agents such as ketoconazole or itraconazole, enzymes such as pancreatin, levodopa, didanosine, pravastatin or digoxin, proteins or peptides such as insulin.

5) Enteric coated stable oral pharmaceutical composition as claimed in claim 1 which is in the form of particles, pellets, granules or tablets or capsules containing the enteric coated particles, pellets or granules.

6) Process for preparing enteric coated stable oral pharmaceutical composition of acid unstable drug comprising formulating the acid unstable drug with pharmaceutically acceptable excipients followed by enteric coating the formulation with an enteric material to provide a bilayer with a pH gradient by first carrying out the enteric coating at neutral or near neutral pH of 7-7.5 to form an inner layer of neutral or near neutral pH and then carrying out the enteric coating at acidic pH of 2-6 to form an outer layer of acidic pH.

7) Process as claimed in clan 6, wherein the first enteric coating at neutral or near neutral pH is carried out to form an inner layer up to ¼th of the enteric coating thickness and the subsequent enteric coating at acidic pH is carried out to form an outer layer up to ¾th of the enteric coating thickness.

8) Process as claimed in claim 6, wherein the enteric coating at neutral or near neutral pH is carried out using an aqueous organic dispersion of the enteric material comprising water and water miscible organic solvent in the ratio 10:90-5:95 v/v and 4-10% by weight of the enteric material.

9) Process as claimed in claim 6, wherein the enteric coating at neutral or near neutral pH is carried out using aqueous organic dispersion of the enteric material comprising water aid isopropyl alcohol in the ratio 8:92 v/v and 6% by weight of the enteric material comprising methacrylate copolymer Type C USP/NF.

10) Process as claimed in claim 6, wherein the enteric coating at acidic pH is carried out using an aqueous dispersion of the enteric material comprising 6 to 10% by weight of the enteric material.

11) Process as claimed in claim 6, wherein the enteric coating at acidic pH is carried out using an aqueous dispersion of the enteric material comprising 8% by weight of methacrylate copolymer Type C USP/NF.

12) Process as claimed in claim 6, wherein the acidic pH is adjusted with sodium hydroxde.

13) Process as claimed in claim 6, wherein the acid unstable drug is benzimidazole derivatives such as omeprazole, pantoprazole, lansoprazole or rabeprazole or salts or optical isomers/enantiomers thereof.

14) Process as claimed in claim 6, wherein the acid unstable drug is penicillin, methicillin, erythromycin and its derivatives, carbenicillin, antifungal agents such as ketoconazole or itraconazole, enzymes such as pancreatin, levodopa, didanosine, pravastatin or digoxin, proteins or peptides such as insulin.

15) Process as claimed in claim 6, which comprises formulating the acid unstable drug with pharmaceutically acceptable excipients in the form of particles, pellets, granules or tablets.
